(19) United States
(12) Patent Application Publication
(10) Pub. No.: US 2014/0100174 A1
(43) Pub. Date: Apr. 10, 2014

(54) Treatment for Cancer

(71) Applicant: Kingsley Yianomah Quartey, Toronto (CA)

(72) Inventor: Kingsley Yianomah Quartey, Toronto (CA)

(21) Appl. No.: 13/645,412

(22) Filed: Oct. 4, 2012

(51) Int. Cl. A61K 31/52; A61P 35/00; A61K 31/7076

(52) U.S. Cl. 514/23; 514/263.23; 514/263.4

(57) ABSTRACT

Treatment for Cancer Using A Preparation containing a combination of one or more Natural or Synthetic or a Combination of Natural and Synthetic Boswellic Acids and one or more Natural or Synthetic or a Combination of Natural and Synthetic Cytokinins such as Kinetin, Kinetin Riboside and other Cytokinins and (the Preparation) could also contain one or more Natural or Synthetic or a Combination of Natural and Synthetic plant growth hormones such as Auxins in any percentage ratio for Treating Cancer.

The said Components of the said Preperation works by Inducing Apoptosis of the Cancerous cells, by “executing” them by Inducing Caspases-8 and other mechanisms.

With the Said Preparation being administered in a Mode or dosage that the Doctor and or Healthcare Practitioner would Prescribe

A Preparation containing a combination of one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.

The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins in any percentage ratio helps Treat and Cure Cancer by “Cell Modulation of the Cancerous Cells”—which “corrects” problems in the Cancerous Cells and problems with the Cancerous Cells and Inducing Apoptosis of the Cancer Cells, by Mechanisms that includes Inducing Caspases, especially Caspase-8, which is essential in Apoptosis of the Cancer Cells.

With the said Preparation being Administered in a Therapeutically Acceptable Dosage, in a Therapeuticaly acceptable Mode and or manner, example, such as orally, or intravenously, and etcetera, with the mode and or method and of dosing and dosage depending on the Health and or Physical and Mental condition of the Patient being treated, the Health Care Practitioner and or the Doctor Prescribing the Said Preparation made with and or Containing one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.

The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins With the said Preparation being Administered in a Therapeuticaly Acceptable Dosage, in a Therapeuticaly acceptable Mode and or manner, example, such as orally, or intravenously, and etcetera, with the mode and or method and of dosing and dosage depending on the Health and or Physical and Mental condition of the Patient being treated.

### BACKGROUND OF INVENTION

### Prior Art

### Cancer

Prior to my Invention, there was no effective Treatment or cure for Cancer.

That was effective as a Treatment or Cure for Cancer.

### Cancer:

Cancer is the uncontrolled growth of abnormal cells in the body. Cancerous cells are also called Malignant Cells.

Cancer usually grows out of normals cells in the body, and Cancer appears to occur when the growth of the cells in the body is out of control and cells divides too quickly.

Cancer can also occur when the cells forget how to die, so more and more cells are occupying the same are without any of the Cancerous cells dying or dying fast enough.

### Types of Cancers:

There are many different kinds of Cancers, and cancer can develop in almost any organ or tissue, such as Lung, Breast, Skin, Bones, Nerve Tissue and others.

There are many types of cancers and Some types are:

Brain Cancer, Breast Cancer, Cervical Cancer, Colon Cancer, Hodgkin's Lymphoma, Leukemia, Prostate Cancer, Kidney Cancer, Liver Cancer, Non-Hodgkin's Lymphoma, Skin Cancer and other forms of Cancers.

### Causes of Cancers:

There are many Causes of Cancers, Including:

Genetics pre-disposition, Environmental Toxins, Radiation Exposure, Certain Viruses, Excessive Consumption Alcohol, and Obesity are all known causes of cancers.

However, there are other causes of Cancers that are unknown

### Prior Art (Treatment of Cancers):

Prior to my My Novel Invention, Treatment of the Various Cancers varies and was based on the type of Cancer that was being treated and the Stage that it was in.

The stage of Cancers refers to ow much the cancer has grown, and whether the cancerous Tumor has spread from its original location.

If the Cancer is confined to one location and has not spread, the most common treatment is if feasible is surgery.

If the Cancer or Cancerous Tumors cannot all be removed by SURGERY, some of the treatment options includes RADIATION, CHEMOTHERAPY, or a Combination of all three.

### Limitations of Current Treatment of Cancers:

### Surgery:

Surgery is Helpful in Treating Cancer, and many times a Surgery can remove all or most of the Cancerous Tumors in those that have Cancer, However, there are times when some of the Cancerous has spread to areas that would cause more harm if any attempt is made to remove those tumors Surgically. There are times too that Surgery do not get all the affected Cancer Cells so even after Surgery the Cancer Tumor grows again.

### Radiation:

Radiation as Treatment for Cancer is usually painless.

However, there are many unpleasant side effects and moderate to severe side effects associated with Radiation Treatment of Cancer patients, and the side effects includes but not limited to “killing of healthy as well as cancerous cells” and the using Radiation to Treat Cancer does not always work.

### Chemotherapy:

Chemotherapy as Treatment for cancer uses a combination of antineoplastic drugs or a combination of antineoplastic drugs and other drugs.

The most common Chemotherapy agents act by killing Cells that divides too rapidly, which is how Cancer cells behaves or divides too rapidly, unfortunately some normals cells such cells in the bone marrow, digestive tracts and hair follicles also divides too rapidly under normal circumstances, meaning, as the chemotherapy tries to kill the cancerous cells by targeting the cells that are diving rapidly, it also end up killing good cells that are also dividing rapidly, thus suppressing the immune system of those being treated for Cancer.

Also Chemotherapy while a good cancer Treatment, it does not always work as a cancer Treatment.

### BRIEF SUMMARY OF THE INVENTION

### Treatment for Cancer

A Preparation containing a combination of one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.

The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins

the Said Preparation made with and or Containing one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer any kind of Cancer, including but not limited to: Brain Cancer, Breast Cancer, Cervical Cancer, Colon Cancer, Hodgkin's Lymphoma, Leukemia, Prostate Cancer, Kidney Cancer, Liver Cancer, Non-Hodgkin's Lymphoma, Skin Cancer and other forms of Cancers

### The Basic Mechanism of How My Invention Works:

The said Preparation for Treating Cancer containing a combination of one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer works in Tandem by “Cell Modulation”—which “corrects” problems in and with the Cancerous Cells and by Inducing Apoptosis or Inducing Death of the Cancer cells without Harming the Normal Cells.

While the one or more Boswellic Acids and one or more Cytokinins such as Kinetin, Kinetin Riboside and other Cytokinins works with or without the Auxins, I believe that a Combination of all three (one or more Boswellic Acids and one or more Cytokinins such as Kinetin, Kinetin Riboside and other Cytokinins works with or without the Auxins) in any percentage ratio helps kill the Cancerous Cells by “Cell Modulation”—which “corrects” problems in and with the Cancerous Cells and Inducing Apoptosis of the Cancer Cells, by Mechanism that includes Inducing Caspases, especially Caspase-8, which is essential in Apoptosis of the Cancer Cells.

Since the failure of Apoptosis of the Cancerous Cells is the Cause of Cancer, by Inducing Caspases, which are often termed as “executioner of cells”, the Caspases and yet to be understand mechanisms “programs the death of the Cancerous cells” and the Cancerous Cells dies off and there is no more Cancer.

### DETAILED DESCRIPTION OF THE INVENTION

### Treatment for Cancer

### Novel Cancer Treatment:

My Novel Invention of A Preparation containing a combination of one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.

The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins

### Treatment for Cancer

Since Cancer is the uncontrolled growth of abnormal cells in the body.

Cancer usually grows out of normals cells in the body, and Cancer appears to occur when the growth of the cells in the body is out of control and cells divides too quickly.

Cancer can also occur when the cells forget how to die, so more and more cells are occupying the same are without any of the Cancerous cells dying or dying fast enough.

To kill Cancerous Cells or Cancerous Tumors, there are number of Tools and or Therapies available to Doctors and Healthcare Practitioners has number of Cancer Treatment Options available to them, including: Surgery, Radiation Therapy, and Chemotherapy.

Unfortunately none of this options are adequate, and sometimes not only do they not work as well as is expected or hoped to work, but they also all have their limitations and sometimes undesirable side effects that could be moderate or severe enough to cause serious health hazard.

By Combining one or more Boswellic Acids, which could be natural or synthetic or a combination of natural and synthetic Boswellic Acids and one or more Natural or Synthetic in a Combination of Natural and Synthetic Cytokinins such as Kinetin, Kinetin Riboside and other Cytokinins or and one or more Natural or Synthetic or a Combination of Natural and Synthetic plant growth hormones such as Auxins in any percentage ratio in a Preperation for Treating Cancer works by killing the Cancerous Cells by Inducing Apoptosis of the Cancer Cells, by Mechanism that includes Inducing Caspases, especially Caspase-8, which is essential in Apoptosis or Death of the Cancer Cells.

Since the failure of Apoptosis of the Cancerous Cells is the Cause of Cancer, by Inducing Caspases, which are often termed as “executioner of cells”, the Caspases “programs the death of the Cancerous cells” and the Cancerous Cells dies off and there is no more Cancer.

1. Using a Preparation containing a combination of one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.
The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins
With the said Preparation being Administered in a Therapeuticaly Acceptable Dosage, in a Therapeuticaly acceptable Mode and or manner, example, such as orally, or intravenously, and etcetera, with the mode and or method and of dosing and dosage depending on the Health and or Physical and Mental condition of the Patient being treated, the Health Care Practitioner and or the Doctor Prescribing the Said Preparation made with and or Containing one or more Boswellic Acids and one or more Cytokinins such as Kinetin, Kinetin Riboside and other Cytokinins and (the Preperation) could also contain one or more plant growth hormones such as Auxins in any percentage ratio for Treating Cancer.
The Boswellic Acids could be Derived from Boswellia Sereta (the Frankincense Tree) and or Extracted from the Resin of the Boswellia Sereta (the Frankincense Tree), or any part or exudate of Boswellia Sereta (the Frankincense Tree) or from any other plant or animal, or it could be Synthetic Man-made, or a combination of Synthetic and Natural Boswellic Acids and the Cytokinins such as Kinetin, Kinetin Riboside and other Cytokinins could be Natural Cytokinins derived from natural source such as any part whatsoever of of the Coconut Tree (Cocos Nucifera or derived and or extracted of the Coconut Tree (Cocos Nucifera), or any plant or animal derived, or a combination of both plant and animal derived and extracted Cytokinins and the Auxin could be Natural derived from any plant or animal and or synthetic Auxins and or a Combination of Synthetic and Natural Auxins With the said Preparation being Administered in a Therapeuticaly Acceptable Dosage, in a Therapeuticaly acceptable Mode and or manner, example, such as orally, or intravenously, and etcetera, with the mode and or method and of dosing and dosage depending on the Health and or Physical and Mental condition of the Patient being treated As my Invention.
